(19) United States
(12) Patent Application Publication
(10) Pub. No.: US 2006/0074285 A1
(43) Pub. Date: Apr. 6, 2006

(54) Apparatus and method for fusion and in-operating-room presentation of volumetric data and 3-D angiographic data

(75) Inventors: Michael Zarkh, Giv'at Shmuel (IL); Omer Barlev, Kohav Yair (IL); Moshe Klaiman, Gedera (IL)

Correspondence Address: DAVIDSON, DAVIDSON & KAPPEL, LLC, 485 SEVENTH AVENUE, 14TH FLOOR, NEW YORK, NY, 10018, US

(73) Assignee: Paieon Inc., New York, NY (US)

(21) Appl. No.: 10/949,155

(22) Filed: Sep. 24, 2004

(51) Int. Cl. A61B 5/05

(52) U.S. Cl. 600/407

(57) ABSTRACT

An apparatus and method for fusing images, views and data acquired prior to a medical operation by a medical imaging device with images, views and data acquired by another medical imaging device during the operation. The acquired and fused data includes identification and classification of plaque deposited along blood vessels.

### BACKGROUND OF THE INVENTION

1. Field of the Invention

The present invention relates to medical imaging systems in general, and to an apparatus and method for presenting in-room, real-time updated 3-dimensional arteries model, including plaque.

2. Discussion of the Related Art

Medical imaging devices are widely used for a number of purposes, both prior to and during medical operations. Pre-operation purposes include evaluation of a patient's status, assessment of required treatment, treatment planning in general and catheterization in particular. During-operation purposes include on-going assessment of the patient's condition, and locating the exact position of invasive tools and devices.

Any currently existing imaging modality has its strengths and weaknesses. Angiograms have high resolution, which enables to depict small vessels (with diameter smaller than 0.8 mm), unnoticeable in other modalities. The high resolution of angiograms and three-dimensional products of angiograms processing, provide accurate measurements of distances, such as arteries diameter. Angiograms are also up-to-date in nature. However, soft tissues are not visible in angiograms so a lot of information, and plaque information in particular is missing when coming to describe in details the current state of arteries.

CT scanners, on the other hand, provide volumetric information and therefore offer 3-dimensional presentation of segmented information, including soft tissues in general and plaque deposited along the arteries in particular. However, CT scans lack up-to-date information, since they are taken prior to an operation. In addition, the resolution of CTs is inferior to the resolution provided by angiograms.

CT scans enable the reconstruction of the vessels structure by tracking the lumen, i.e., the space inside the arteries. The shortcoming of this construction is that if a blood vessel is blocked, little or no blood flows through it and the relevant part of the blood vessel can not be visually reached by tracking the lumen. In addition, the blood vessels' walls can be viewed in CT scans provided they are at least 1.5 mm wide (the width of a healthy coronary artery, for example, is 0.1-0.9 mm). Therefore, it might be impossible to tell that a blood vessel carries significant sediments until it is substantially damaged, or to assess the percentage of the stenosis of the blood vessel. Using the standard tools, it is only possible to tell if stenosis takes up more or less than 50% of the vessel's diameter. If the vessel wall is 2mm or greater, a more precise estimation of the percentage of the stenosis can be provided.

There is therefore a need in the art for a system that will combine the best of CT with the best of angiograms to supply in-operating-room, up-to-date, accurate 3-dimensional information.

### SUMMARY OF THE PRESENT INVENTION

In accordance with a first aspect of the present invention, there is provided an apparatus for displaying a first image, said first image is a product of processing a second image taken by a medical imaging device prior to a medical operation, the first image comprising information about areas having sediments, wherein during the operation, said first image is presented to a user of the system. The first image is fused with a third image said third image is a product of processing a fourth image taken by a medical imaging device during the operation. The apparatus further comprises a computer program for registration of images of the medical imaging devices; and fusing information contained in and associated with products of processing images of the medical imaging devices; and presenting the first or third or a combination of the first and third images containing information obtained from the medical imaging devices or an image containing information obtained from the either one of the medical imaging devices. The apparatus also comprising a correction module to correct imaging errors in the second image, using the fourth image acquired during the operation. The imaging error is characterized by one or more calcified areas of one or more blood vessels depicted outsized on an image, said image is a product of processing of one or more images taken by one or more medical imaging device prior to an operation. In the preferred embodiment the first image and the third image, are presented on the same location on the visual display, where the first and the third images are partially transparent. At least a part of the first image, and at least a part of the third image can be presented adjacent to each other. Sediments found by processing the first image are distinctively marked on the third image. The sediments found by processing the first image are distinctively marked on the first image. The apparatus further comprising a marking module for marking one non-flexible part of the blood vessel, on the first or third image. The apparatus further comprising a module for marking the at least one curved part of the blood vessel, on the first or third image. The apparatus further comprises a module for marking indications prepared prior to the operation, on the first or third image. The apparatus further comprises a module for indicating during an operation, parameters determined prior to the operation, for a medical imaging device, said parameters to be applied while taking images. The apparatus further comprises a module for: identifying one point in an image presented during the operation with one or more check-points indicated prior to the operation; and presenting the image, associated prior to the operation with the one or more check-points. The blood vessel can be a coronary artery. The sediments can be any one of the following: lipid-rich plaque, intermediate plaque, calcified plaque, thrombi, cells or products of cells. The medical imaging device can be a multi slice computerized tomography device.

In accordance with a second aspect of the present invention there is provided an apparatus for detecting a part of a blood vessel with sediments, from a first image acquired by an imaging device prior to an operation, the apparatus comprises: an identification module for identifying the part of the blood vessel, and within said part the sediments located therein; and a marking module for indicating the part of the blood vessel and sediment associated therewith on a second image created by processing images taken by a medical imaging device. The identification module is receiving intensity values for the pixel of the image acquired by a medical imaging device; and range of intensity values for the type of sediment. The apparatus further comprises a module for constructing a visual representation of the lumen of the blood vessel. The apparatus further comprising a module for constructing a visual representation of the part of the wall of the blood vessel. The parts of the blood vessel and the sediment submerged therein are indicated using color-coding. The apparatus further comprising a width determination module for determining the width of the sediment layers at a location along a blood vessel; and the diameter of a blood vessel at a location along the blood vessel; and the percentage of stenosis of a blood vessel at a location along the blood vessel. The widths of the sediment layers, the diameter of the blood vessel and the percentage of stenosis are indicated on the second image. The apparatus further comprises a module for indicating, in response to a user action, a part of the blood vessel as non-flexible. The apparatus further enabling comprises a module for indicating, in response to a user action, a part of the blood vessel as curved. The apparatus further comprises a check-point definition module for indicating, in response to a user action, a position within the body of a patient as a check-point and associate said check-point with the image produced by processing the first image. The second image depicts a three-dimensional view of a part of the blood vessel. The second image depicts a surface within the human body and the blood vessel on said surface. The second image depicts an internal three-dimensional view of the blood vessel. The second image depicts a cross-section of a blood vessel at a location along the blood vessel, said cross-section comprising one or more of the following: the blood vessel's wall, the lumen of the blood vessel, sediments submerged on the blood vessel's wall. The apparatus further comprises a module for manually correcting the indications for sediments on images acquired prior to an operation and the products of said images. The correction includes changing the size or the sediment type of an indication, adding, or deleting indications.

In accordance with a third aspect of the present invention there is provided a method for displaying a first image, said first image is a product of processing a second image taken by a medical imaging device prior to a medical operation, the image comprising information about areas with sediments, during the operation. The first image is fused with a third image which is a product of processing a fourth image taken by a second medical imaging device during the operation, the method comprises the following steps: registering the coordinate systems of the first and second medical imaging devices; and fusing information contained in and associated with products of processing images of the first or second medical imaging devices; and presenting an image from the first or second medical imaging devices or an image containing information of images from the first and second medical imaging devices. The registration of the coordinate systems comprises the following steps: matching three or more points seen in images of the first and second medical imaging devices; and matching the coordinate frames of the first and second medical imaging devices. The matching of the points is based on comparing the coordinates of three or more non-aligned fiducials as seen in the image of each of the two medical imaging devices. The matching of the three points is based on comparing an at least one two-dimensional image taken during an operation to an at least one projection of a three dimensional image constructed from at least two two-dimensional images taken by an at least one medical imaging device prior to the operation. The method further comprising a step of correcting an imaging error of the image taken by a medical imaging device prior to an operation, using the image acquired during the operation. The imaging error is characterized by a calcified area of a blood vessel depicted outsized on one or more products of processing of the image taken by a medical imaging device prior to an operation. The registration of the coordinate systems comprises the following steps global registration of a first image and a second image taken by the first and the second medical imaging devices; and removal of local residual discrepancies by matching corresponding features detected in the first and the second images. The global registration is based on comparing the coordinates of a fiducial as seen in the first and the second images. The global registration is based on matching one or more two-dimensional images taken during an operation to a projection of a three dimensional data obtained from the medical imaging device prior to the operation. The first image and the third image, are presented on the same location on the visual display, where the first and the third images are at least partially transparent. The method further comprising a step of marking non-flexible part of a blood vessel, on a first image. The method further comprising a step of marking non-flexible part of a blood vessel, on the first image. The method further comprising a step of marking curved portion of the blood vessel, on first image. The method further comprising a step of marking curved portion of the blood vessel, on the third image. The method further comprising the steps of identifying a point in an image acquired during the operation with a check-point indicated prior to the operation; and presenting the image associated prior to the operation with the check-point.

In accordance with a fourth aspect of the present invention there is provided a method for automatic reconstruction of a three-dimensional objects from two angiograms using CT information, the method comprises the following steps taking a first and a second angiograms of the required area from different perspectives; and for the first and the second angiogram, obtaining a first and a second projected CT images by projecting the three dimensional CT data on the same plane as the first and the second angiogram; and registration of the first and the second angiogram with the corresponding projected CT images by objects appearing in the angiogram and in the projected CT; and mutual co-registration of the first and the second angiograms; and detecting objects appearing in the angiogram and match with the corresponding objects in the projected CT; and deriving the three dimensional coordinates of the objects appearing in the first and the second angiograms; and constructing a three dimensional image of the required area from the first and the second angiogram.

### BRIEF DESCRIPTION OF THE DRAWINGS

The present invention will be understood and appreciated more fully from the following detailed description taken in conjunction with the drawings in which:

FIG. 1 is a schematic block diagram of the proposed apparatus, in accordance with the preferred embodiment of the present invention;

FIG. 2 is a schematic block diagram of the operating components of the pre-operation modules, in accordance with the preferred embodiment of the present invention;

FIG. 3 is a schematic block diagram of the image fusion method.

FIG. 4 is a schematic block diagram of the operating components of the during-operation modules, in accordance with the preferred embodiment of the present invention;

### DETAILED DESCRIPTION OF THE PREFERRED EMBODIMENT

An apparatus and method for fusing images and information about tubular organs, from CT scans and angiograms, and presentation of the same in 3-dimensions during medical operations is disclosed. The presented information includes different types of sediments deposited inside and outside coronary arteries or other blood vessels, as part of the whole structure of the blood vessels. The apparatus is designed to be used both before and during a medical operation, usually a catheterization, and also to enable the user to mark different areas of interest and pre-defined views prior to the operation. The areas and views will be presented by the system during an operation.

The preferred embodiment of this invention uses slices taken by a Multi-Slice Computerized Tomography (MSCT) device. The MSCT scanner can simultaneously acquire up to 32, 40, or even 64 slices, thus covering the whole heart area by slices 0.6 mm apart, that were taken during a time frame of 10-20 seconds. Therefore, the scanner enables high-resolution morphologic evaluation of the myocardium and the coronary arteries as well as of other blood vessels. The MSCT yields a pixel size of 0.3-0.5 mm and temporal resolution of 90-120 mSec.

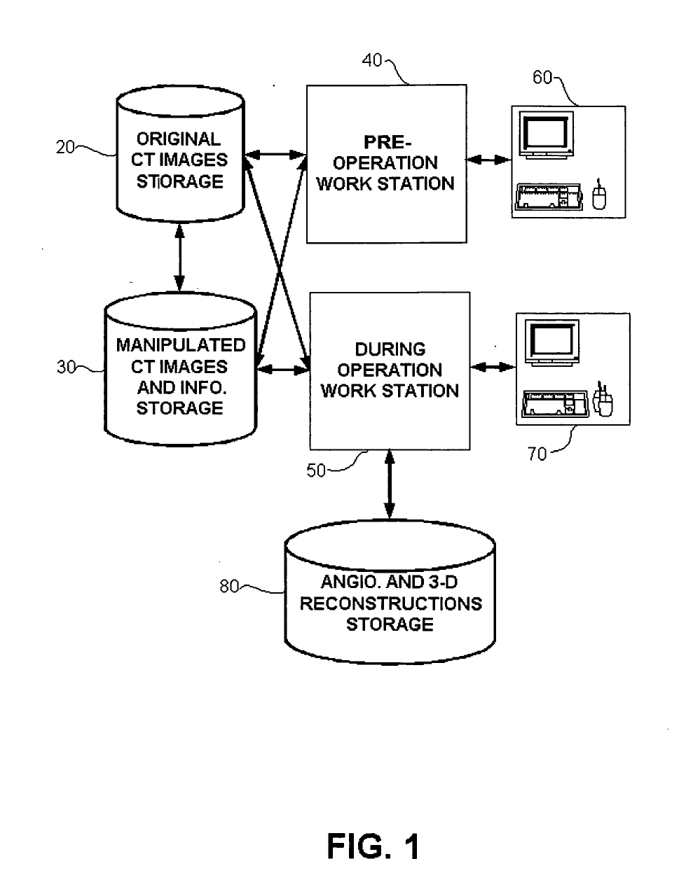

Referring now to FIG. 1 that shows an exemplary environment in which the proposed apparatus and associated methods are implemented. In the present non-limiting example, the environment is a cardiologic department of a health care institute. A patient who is suspected (or known) to suffer from a coronary arteries problem, or another problem related to sediments on blood vessels, goes through a scanning session by a medical imaging device. A possible conclusion of the physician evaluating the images taken by the device is that the patient should be catheterized. In this case, the proposed invention discloses how to fuse and present images taken or generated prior to the operation, with images taken or generated during the operation. When relating to images taken prior to or during an operation, the discussion includes both the images as taken, and products of processing the taken images. The products are model of different body parts or body tissues, such as a vascular tree, a heart muscle, plaque or the like. The structures may be described as collections of volume elements, tubular organs given by lines and radii, surface and so on. The mentioned structures, of course, are associated with one or more visual presentations.

In the framework of this exemplary system, the pre-operation input to the system comprises images of a body part, for example the heart area of patient, taken by an MSCT scanner (not shown). The original images, as scanned by the MSCT (also referred to as slices) are stored on a storage device 20. The additional images and information storage 30 stores images and other information produced by processing the original images. This processing is initiated by the user's actions and is performed by the pre-operation work station 40. The angiogram and the 3-dimensional reconstructions of vessels based on the angiograms, are used during the operation by the during-operation work station 50 and are stored in the angiogram and 3-dimensional reconstruction storage 80. Each of the pre-operation work station 40 and the during-operation work station 50 is preferably a computing platform, such as a personal computer, a mainframe computer, or any other type of computing platform that is provisioned with a memory device, a CPU or microprocessor device (not shown), and several I/O ports (not shown). Alternatively, the pre-operation work station 40 and the during-operation work station 50 can be DSP chips, ASIC devices storing the commands and data necessary to execute the methods of the present invention, or the like. The pre-operation work station 40 and the during-operation work-station 50 are further equipped with standard means for collecting input from the user and presenting the results 60 and 70 correspondingly. In the exemplary environment of the present application, these would usually comprise a keyboard, a pointing device such as a mouse, and a display device. The pre-operation work station 40 and the during-operation work-station 50 can further include internal storage devices (not shown), storing the computer applications associated with the present invention. These storage devices can also serve as the original images storage 20, the additional images and information storage 30 and the angiograms and 3-dimensional reconstruction storage 80. The storage units 20, 30 and 80 can be magnetic tapes, magnetic discs, optical discs, laser discs, mass-storage devices, or the like. The computer application associated with the present invention is a set of logically inter-related computer programs and associated data structures that interact to perform the tasks detailed hereinafter. The pre-operation work station 40 and the during-operation work station 50 can be the same machine, separate machines and even different machines. Optionally, the pre-operation work station 40 and the during-operation work station obtain the original images, or store and obtain the images and information from sources other than the original image storage 20, the manipulated images and info storage 30, and the angiograms and 3-dimensional reconstruction storage 80, such as a remote source, a remote or local network, a satellite, a floppy disc, a removable device and the like.

Further note should be taken that the apparatus presented is exemplary only. In other preferred embodiments of the present invention, the computer applications, the original images storage 20, the manipulated images and additional information storage 30, the angiograms and 3-dimensional reconstructions storage 80, the pre-operation work station 40 and the during operation work station 50 could be co-located on the same computing platform. As a result, one of the I/O sets 60 or 70 will be rendered unnecessary.

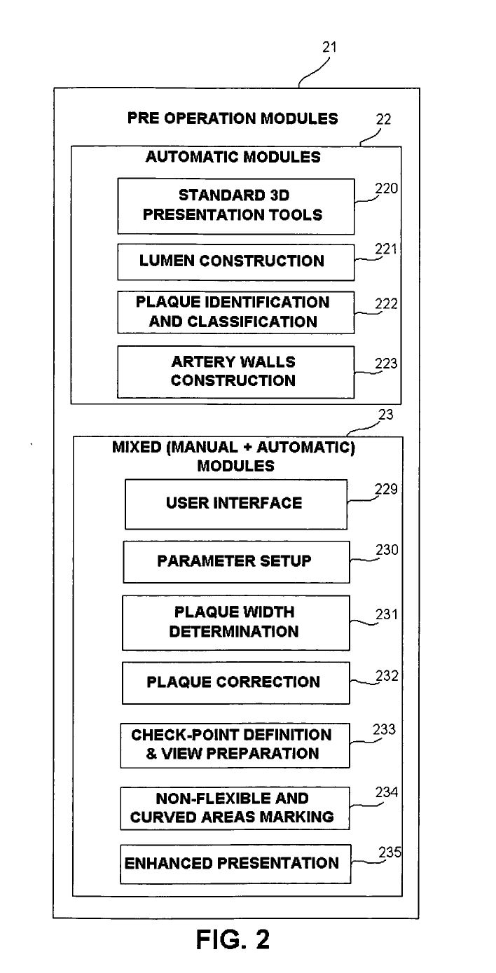

Referring now to FIG. 2 that shows the various modules that perform actions on the MSCT images, prior to an operation. It is important to get a good understanding of the features and tools available in the pre-operation stage, because the products of these tools are used in the during-operation stage detailed below. The pre-operation modules are divided into automatic modules 22 that do not require user interaction during their work, and mixed modules 23, in which the system executes commands in response to the user's actions and inputs. The user is typically a physician or a skilled technician. This division to automatic and mixed tools is for clarity reasons only, and does not imply order of activation, precedence or the like. The modules to be activated and their order depend on the user's choice. In addition, software engineering considerations might cause some functionality of certain modules to be called automatically from other modules. The products of all the modules of FIG. 2 are stores in the additional images and information storage 30 of FIG. 1.

The automatic modules 22 comprise a number of inter-related computer implemented modules. The standard 3-D presentation tools module 220 can be a computer program for presenting 3-D images, provided by the MSCT manufactures and also by independent manufactures such as VITREA® manufactured by Vital Images, Plymouth, Minn., USA. The data acquired by CT scanners is volumetric in nature, i.e. intensity information is associated with each volume unit, named voxel. Since each substance scanned has a specific range of intensity, the intensity data represents the composition of the area scanned. The CT intensity is measured in Hounsfield Units (HU). This raw volumetric information enables the reconstruction of segmented information, i.e. reconstruction of specific body parts and tissues.

The presentation tools 220 enable a number of processing and viewing options of the scanned images.

One option is to show visible two-dimensional or three-dimensional presentations of the models of various body parts and tissues, the models constructed from at least two slices scanned by the device. Two-dimensional images include, for example, scanned slices. Three-dimensional images include, for example, images of surfaces, images of an arteries structure and the like. Another presentation option involves producing new planar images, either parallel or at a predetermined angle to slices taken by the imaging device. Yet another option is presenting a cross section of an artery, or even a sequence of such cross sections, thus visualizing “fly through”.

One more option is to present one or more images in various layouts, such as presenting at least two adjacent images depicting the same or adjacent locations, presenting images in a temporal sequence, and the like.

The above mentioned views, images, and their combinations are stored in the additional images and information storage 30 of FIG. 1.

The lumen construction tool 221 can also be a computer program for constructing 3D images showing lumen, such as VITREA® manufactured by Vital Images, Plymouth, Minn., USA. The tool 221 reconstructs the vessels structure by tracking the lumen, i.e., the space inside the arteries. As mentioned earlier, blocked parts of small diameter vessels are unreachable.

The plaque identification and classification module 223 identifies the various types of plaque that might be deposited in the blood vessels. This is enabled by the high spatial and temporal resolution of the MSCT device, relatively to single-slice CT scanners (the angiograms, although high-resolution, do not enable soft-tissues imaging). The detection of the sediment type is performed by traversing the data structure representing the lumen and comparing the intensity data associated with the area adjacent to the lumen with predefined intensity ranges. Thus, the detection of sediment is performed by “tracking” the blood vessels through the lumen structure constructed by the lumen construction module 221 as a road map, and comparing the values of the CT intensity found in the vicinity of the blood vessels to known ranges (after ignoring the values associated with the heart surface). The following table lists exemplary ranges of values for each type of sediment:

| Sediment Type | Intensity (HU) |
|---|---|
| Lipid-rich (soft) plaque | >=12 and <=62 |
| Fibroid (intermediate) plaque | 50-130 |
| Calcified plaque | 120<= |

The table was presented by Stephen Schroder, Tubingen, at the “International Task Force for Prevention of Coronary Heart Disease” Symposium, Scuol, Feb. 23, 2003.

As can be noticed from the table, some ranges of intensity can be interpreted in more than one mode. In such cases considerations of continuity with surrounding areas will be applied.

The blood vessels' walls construction module 223 retrieves the information about the blood vessels' walls that can be deduced from the high-resolution CT slices. When using the MSCT technology, each pixel represents a square with a side of 0.4mm in average. Due to rounding problems, at least two pixels are required in order to detect an edge in general and the wall of the blood vessel in particular. Thus, only blood vessels' walls that are at least 0.8mm wide can be recognized accurately. For blood vessels with thinner walls, rounding problems cause substantial errors and inhibit the correct presentation. The information about the lumen, sediments, and vessels' walls combined together, provides an informative view of the blood vessels, and is stored in the additional images and information storage 30 of FIG. 1.

The mixed (automatic and manual) modules 23 comprise a number of inter-related computer implemented modules. The user interface module 229 presents the user with all the options he or she can chose from when working with the system. The presentation of these options uses graphic, textual or any other means. When choosing a certain option, the system enables the user to make the relevant choices, perform the relevant actions and store the results. For example, when users select the “check point definition” option, the system would allow the user to define a check-point and associate views therewith as is explained below in the description of the check-point definition and view preparation module 233.

The parameter setup module 230 is used for setting system parameters and user preferences, such as color selection, preferred layouts of images, numerical parameters and the like. Such parameters are used by both the automatic and the mixed tools, both prior to and during the operation. The parameters and settings are stored in the additional images and information storage 30 of FIG. 1.

The plaque width calculation module 231 enables the user to point at a specific location along a blood vessel, and have the system calculate the actual width of the plaque layers deposited at the location; the actual width of the lumen at that location; and the percentage of stenosis, if any, at that location. The stenosis percentage is determined by 1 minus the ratio between the actual area of the cross section of the artery at the required location and the average area of the cross section along the artery. This average is determined from the graph representing the cross-section's area distally and proximally from the required location. All mentioned information—the plaque width, the blood vessel's width and the percentage of stenosis are stored in the additional images and information storage 30 of FIG. 1.

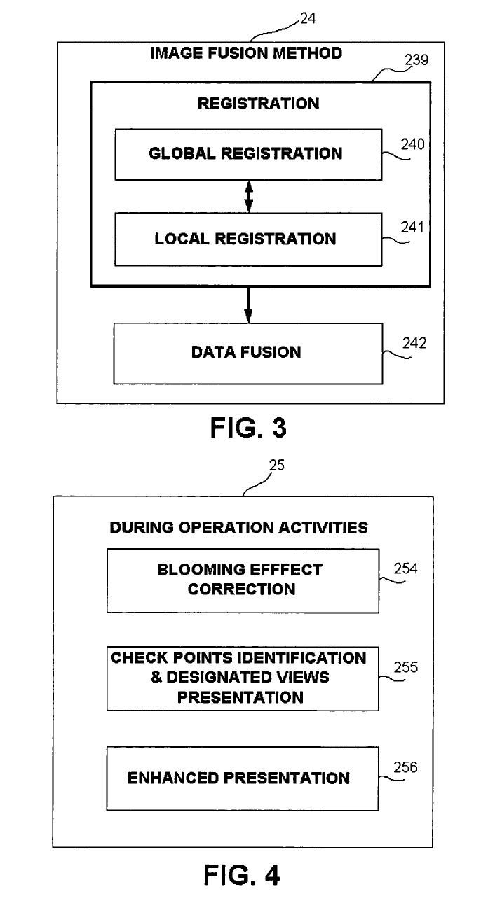

The plaque correction module 232 enables the user to manually change the type, size, density and shape of any plaque sediment recognized by the system. It also enables the user to add or remove indications for plaque. In particular, correction might be needed in areas suffering from the blooming effect, due to which heavily calcified areas appear outsized. This is caused by the reflection of x-rays from the calcified areas onto their neighboring areas. This effect is automatically corrected in the during-operation system in the blooming effect correction module 254 of FIG. 3. This automatic correction can not be performed without additional images of the area, such as angiograms and 3-dimensional reconstructions from angiograms, which are usually available only during the operation. A method for performing three-dimensional reconstruction is disclosed, for example, in U.S. Provisional Application No. 60/577,981 assigned to the assignee of the present invention, incorporated herein by reference. The user input is accepted through the use of the keyboard and the pointing device 60 of FIG. 1. The corrections to the plaque areas are stored in the additional images and information storage 30 of FIG. 1.

In the check-point definition and view preparation module 233 the user can designate any point in the heart area of the patient as a check-point. Since all acquired data carries volumetric information, each pixel seen in an original slice or on certain types of derived images can be uniquely identified with the corresponding location in the imaged volume. Clicking with the mouse or otherwise pointing at such point defines it as a check-point when the system is at the check- point selection mode. The user can then associate one or more views with each check-point. The views can be originally acquired slices, any other views as described hereinafter in the description of the enhanced presentation module 235, or any combination of the above. The user can also associate recommendations for preferred perspectives of the medical imaging device being used during the operation, for better view of the relevant area. The check-points and the views and recommendations associated with them are stored in the additional images and information storage 30 of FIG. 1. The check-points and associated views are used during the operation as will be explained in the description of the check-point identification and designated views presentation module 256 of FIG. 3.

The non-flexible and curved areas marking module 234 enables the user to mark parts of blood vessels as non-flexible or curved. Due to the volumetric information of the CT data, it is possible to mark a relevant area in an image, that is identify location of desired area in CT volume. The marking can take place on an original slice or volume, or on the visually presented product of processing. The marking is performed, for example, by designating two points along a blood vessel so that the part of the blood vessel between these two locations is marked as non-flexible. In another embodiment the user freely draws the curved line along which the blood vessel is curving. This option is particularly useful in the highly curved areas of the blood vessels, or in areas where blood vessels branch. As with the check-point definition module, the user can associate any desired views with the marked areas. The marked areas, their types and the associated views are stored in the additional images and information storage 30 of FIG. 1.

The enhanced presentation module 235 complements the standard presentation tools module 220. This module presents all the additional information deduced by the system and indicated by the user using the automatic modules 22 and the mixed modules 23, over the views mentioned above in the standard 3-D presentation tools module 220. One type of information included is the marking of the different types of plaque layers as deduced by the system in the plaque identification and classification module 222 and possibly corrected by the user in the plaque correction module 232. Such layers are typically indicated by using a designated color for each type of sediment, selected in the parameter setup module 230. Other data include the designated check-points, and the non-flexible and curved areas of the blood vessels. Yet more data includes the numerical values obtained by the plaque width determination 231, including the width of the various plaque layers, the diameter of the blood vessel at the specified location, and the percentage of stenosis in that location. The previous description relates to the modules and tools available during the pre-operation mode. Following are the methods and modules used during the operation, in order to fuse and present information gathered prior to and during the operation.

In a typical non-limiting environment in which the disclosed invention is used, a patient is scanned by an MSCT imaging device, and the products of the scan are analyzed by a physician or a skilled technician. The results of the analysis can include a decision that the patient does need to undergo a catheterization, and the products of the pre-operation modules as described in FIG. 2.

The previous description relates to the modules and tools available during the pre-operation mode. Following are the methods and modules used during the operation, in order to fuse and present information gathered prior to and during the operation. The pre-operation preparations are optional. All required operations can be performed immediately before or during the operation. Once the catheterization is in progress, the operating physician takes angiograms of the patient. The angiograms are taken at different locations, perspectives and magnifications according to the physician's needs at any given moment during the operation. The angiograms locations and angles can also be determined prior to the operation by a planning system to get best view of the problem. The angiograms undergo processing yielding 3-dimensional reconstructions. The disclosed invention uses images and products of images acquired prior to the operation, during the operation. These images and products are fused with images and products acquired during the operation.

Referring now to FIG. 3 that shows the method used in the proposed invention for fusing the images and products acquired prior to the operation with the images and products acquired during the operation.

The method comprises the following steps:

In step 239 registration is carried out, meaning establishing transformation between objects detected in CT volume and in angiograms.

In step 240, global registration is performed, in which the best set of parameters defining projection of CT volume into angiographic image plane is recovered. The global registration can be carried out in a number of ways. The first way is the use of calibration devices or fiducials. Fiducials are screws or other small objects made of material visible and easily detectible both in the MSCT volume and in the angiograms, such as Titanium. The fiducials are attached to the patient's body and do not change location between the CT imaging and the catheterization procedure, therefore their location in the CT and on the angiogram disclose the transformation between the two coordinate frames. Another way of performing the global registration is by using the parameters supplied by imaging system. Yet another option for the global registration involves the usage of iterative process of imaging parameter recovering utilizing automatic detection of corresponding points in 3-dimensional volume and 2-dimensional projection. One variant of this process comprises the steps of preparing a synthetic image based on projection of CT volume or information extracted from CT volume with approximately known imaging parameters; matching of synthetic image with real angiogram using, for example, correlation technique; refinement of imaging parameters according to the found local displacements between the two images; and repeat the steps until the process converges to the best imaging parameters. Combinations of the abovementioned methods for the global registration can be applied as well.

The global registration process yields for every voxel of CT volume, a unique location in the angiographic image. In the opposite direction, generally every pixel in an angiogram can be mapped into straight line in volume. However, if the pixel belongs to a detected feature in angiogram and is associated with certain voxel of the corresponding feature detected in CT volume, the correspondence for such pixel is then established. Therefore, matching of corresponding features in 3 dimensions and 2 dimensions is an essential part of establishing a bilateral correspondence. Matching the features is possible due to the hierarchy structure and distinctive geometry of blood vessels, i.e. their shapes and intersections. If the blood vessels network was denser, such matching might not have been possible. Alternatively, if two corresponding pixels are identified in two different angiograms then a 3-dimensional location of a corresponding voxel can be also established.

In step 241 a local registration is performed which includes removal of residual discrepancy between corresponding features detected in CT and angiograms. Specifically, the tree of 3-dimensional centerlines of blood vessels extracted from the CT data is matched with the two-dimensional tree extracted from two-dimensional angiograms, including branch-to-branch matching on high level and point-to-point matching within each matched branches on low level. Based on the matching of the trees, the global transformation can be augmented with continuously changing local correction function. This correction allows the establishment of an exact transformation not only for the local features themselves, but also for neighboring areas.

In step 242, the images and detailed information acquired prior to the operation are fused with the most updated visual information as acquired by the angiograms during the operation. The fusion process uses the transformation found in step 239. The data fusion process starts from a three dimensional image created from the CT data. The centerlines of the blood vessels are derived from the CT data. A fused model combine regions with different resolutions. The lumen around the vessel centerlines is presented with its structure derived from the CT and the high-resolution details originating from angiograms, whereas surrounding areas are represented with lower resolution information as acquired by the CT. Data fusion also takes place when presenting a cross-section of an artery. The approximate shape of the cross-section of the artery is known from the CT images, and so are the depositions of plaque. However, since the resolution of the CT is inferior to that of the angio, the vessels boundaries information and numeric data, such as the area of the cross section are fused with the image and enhance it. The lumen area at any location along the vessel (i.e., the area of the cross-section of the blood vessel) is taken from the angio information. When the CT cross-section of the blood vessel at the same location is zoomed in, the transitions between sediments areas around the lumen and the lumen itself are fine-tuned to fit the lumen area as determined by the angio. An important addition of the angio to the image fusion is the detection of small vessels that are not seen in the CT. The 3-dimensional coordinates of these vessels are determined by the 3-dimensional angio system, and thus they are fused with the 3-dimensional CT image.

Referring now to FIG. 4 that shows the options available to a user and method of the present invention during the operation. The activities associated with these options are performed by the during-operation work station 50 of FIG. 1, during a medical operation, 25 typically a catheterization.

When the blooming effect correction option 254 is used, the system corrects the errors caused by the blooming effect, due to which some calcified areas look larger in CT images than they should. The error is correctable since the angiograms do indicate the correct size of the lumen in areas in which the blooming effect in the CT data concealed the lumen.

The check-point identification and designated views presentation option 255, supports using the check-points defined with the pre-operation check-point definition and view preparation module 233 of FIG. 2. Whenever the coordinates of a check-point are included in an image taken by the angiogram, the system automatically indicates the presence of a check-point and presents the views associated with a specific check-point at the pre-operation stage. The presence of a check-point in the current angiogram is determined by checking if the coordinates of the check-point as projected onto the angio plane are within the boundaries of the angio image.

The enhanced presentation option 256 presents all the images and views described in the pre-operation enhanced presentation module 235 of FIG. 2. In addition, up-to-date angio data acquired during the operation is fused with the pre-operation images and views to create high-resolution up-to-date three-dimensional images. In the following sections, describing the advanced presentation methods, it should be noted that when referring to an image, it is either an original image acquired by a device, or a product of processing such images. For CT images, such products include three-dimensional views of vascular trees, surfaces, and the like, plaque indications, check-point indications, measurements and the like. For angio images, the products include measurements, three-dimensional images of vascular trees acquired from multiple angiograms and the like.

In accordance with the preferred embodiment of the present invention, three dimensional fused images are presented, in which the “skeleton” or the geometry of the blood vessels tree, is taken from the CT images, and the exact measurements and high resolution presentation is derived from the angio. Another contribution of the CT images to the fusion is the identification of plaque sediments. The fusion is performed as explained above in step 241 of FIG. 3. Another fusion option involves presenting angio images view of 3-dimensional reconstructions with plaque indications derived from the pre-operation stage. The indicated plaque layers can incorporate the correction of the blooming effect present in the pre-operation stage, by the higher-resolution angiograms. Another example for fused elements is the marking of non-flexible or curved areas of the vessels as defined in the non-flexible and curved areas marking module 234 of FIG. 2, on the angiograms. Yet another example is presenting the plaque layers dimensions, the blood vessel's diameter and the stenosis percentage, as enhanced during the operation.

In accordance with another preferred embodiment of the present invention, images of both devices are viewed side by side. The images can depict the same area of the body, different views of the same body area, partly overlapping body areas or totally non-overlapping body areas. In another preferred embodiment, an image taken by one device depicting a certain area is bordered on one or more sides by one or more images of the other device, depicting areas which are neighboring the area depicted by the image of the first device. The effect of this type of presentation is a continuous view of an area, where certain sub-parts of the area were scanned by one device and the other sub-parts were scanned by a second device. In yet another preferred embodiment, images taken by a first device prior to the operation and images taken by a second device during the operation are presented one on top of the other where the top image is at least partially transparent. In another embodiment an image acquired by one device, and a larger image acquired by another device are presented where the larger image is surrounding the smaller image. The two images can depict the same area of the body, neighboring areas or different areas.

The above shown examples serve merely to provide a clear understanding of the invention and not to limit the scope of the present invention or the claims appended thereto. Persons skilled in the art will appreciate that different or additional modules and methods can be used in association with the present invention so as to meet the invention's goals. In particular, different methods of fusing and different fused elements can be used.

The proposed apparatus and methods are innovative in presenting during an operation, images and products acquired prior to the operation and fusing them with images and data taken during the operation. The apparatus also takes advantage of the developing technology of MSCT devices, which enables identification and classification of sediments in blood vessels in general and the coronary arteries in particular, and assessment of the percentage and shape of stenosis in these blood vessels. This facilitates better assessment of the patient's status and aids in the planning and during the execution of a catheterization.

The proposed apparatus also facilitates the construction of three-dimensional angio images without the interaction of a human operator. This is performed by automatic registration of each angiogram to a two-dimensional projection of the CT data, and identifying objects appearing both in the angiogram and in the CT. This, in turn, allows for exact matching between the two or more angiograms and enables three-dimensional reconstruction from these images.

Persons skilled in the art will appreciate that the present invention can also be used with other modalities, such as MRI, once its resolution and scanning rate enable the identification and classification of plaque. Plaque is identified by MR parameters like T₁, T₂, diffusion coefficient, and other MRI tissue characteristics. It is also possible to use more than one set of images, possibly of different modalities, prior to the operation, and take the advantages of each of them in order to accurately assess the status of the coronaries. For example, one possible combination is a black blood MRI identifying the plaque with bright blood MRI identifying the lumen. Registration of black MRI vs bright MRI is done by using the imager common coordinate system. When fusing MR with CT images, the registration method of MR and CT is well known in the literature.

It will be appreciated by persons skilled in the art that the present invention is not limited to what has been particularly shown and described hereinabove. Rather the scope of the present invention is defined only by the claims which follow.

I/we claim:

1. An apparatus for displaying an at least one first image, said first image is a product of processing an at least one second image taken by a first medical imaging device prior to a medical operation, the first image comprising information about areas having sediments, wherein during the operation, said first image is presented to a user of the system.

2. The apparatus of claim 1 where the at least one first image is fused with an at least one third image said third image is a product of processing an at least one fourth image taken by a second medical imaging device during the operation.

3. The apparatus of claim 2 further comprising a computer program for:
registration of images of the first and second medical imaging devices; and
fusing information contained in and associated with products of processing images of the first and second medical imaging devices; and
presenting an at least one combination image, the combination image is selected from the group consisting of: the at least one first image, the at least one third image, a combination of the at least one first and the at least one third images containing information obtained from the at least one first or the at least one second medical imaging devices, an image containing information obtained from the first and second medical imaging devices.

4. The apparatus of claim 2 further comprising a correction module to correct an at least one imaging error in the at least one second image, using the at least one fourth image acquired during the operation.

5. The apparatus of claim 4 where the imaging error is characterized by an at least one calcified area of an at least one blood vessel depicted outsized on an at least one image, said at least one image is a product of processing an at least one image taken by an at least one medical imaging device prior to an operation.

6. The apparatus of claim 2 where the at least one first image and the at least one third image, are presented on the same location on the visual display, where the at least one first and the at least one third images are at least partially transparent.

7. The apparatus of claim 2 where an at least one part of the at least one first image, and an at least one part of the at least one third image are presented adjacent to each other.

8. The apparatus of claim 2 where sediments found by processing the at least one first image are distinctively marked on the at least one combination image.

9. The apparatus of claim 1 where sediments found by processing the at least one first image are distinctively marked on the at least one first image.

10. The apparatus of claim 1 further comprising a marking module for marking an at least one non-flexible part of an at least one blood vessel, on the at least one first image.

11. The apparatus of claim 2 further comprising a marking module for marking an at least one non-flexible part of an at least one blood vessel, on the at least one combination image.

12. The apparatus of claim 1 further comprising a module for marking at least one curved part of an at least one blood vessel, on the at least one first image.

13. The apparatus of claim 2 further comprising a module for marking at least one curved part of an at least one blood vessel, on the at least one combination image.

14. The apparatus of claim 1 further comprising a module for marking indications prepared prior to the operation, on the at least one first image.

15. The apparatus of claim 2 further comprising a module for marking indications prepared prior to the operation, on the at least one combination image.

16. The apparatus of claim 2 further comprising a module for indicating during an operation, perspectives determined prior to the operation, for a medical imaging device, said perspectives to be used while taking images during the operation.

17. The apparatus of claim 2 further comprising a module for:
identifying at least one point in an at least one image presented during an operation with an at least one check-point indicated prior to the operation; and
presenting at least one image, associated prior to the operation with the at least one check-point;

18. The apparatus of claim 1 further comprising a module for the setup of system and user preferences.

19. The apparatus of claim 1 where the blood vessel is a coronary artery.

20. The apparatus of claim 1 where the sediments are any one of the following: lipid-rich plaque, intermediate plaque, calcified plaque, thrombi, cells or products of cells.

21. The apparatus of claim 1 where the first medical imaging device is a multi slice computerized tomography device.

22. An apparatus for detecting at least one part of at least one blood vessel with sediments layers, from an at least one first image acquired by an at least one imaging device prior to an operation, the apparatus comprises:
an identification module for identifying the at least one part of the at least one blood vessel, and within said part the sediments layers located therein; and
a marking module for indicating the at least one part of the at least one blood vessel and sediment associated therewith on an at least one second image created by processing images taken by a medical imaging device.

23. The apparatus of claim 22 where the identification module is receiving:
intensity values for at least one pixel of at least one image acquired by a medical imaging device; and
at least one range of intensity values for at least one type of sediment.

24. The apparatus of claim 22 further comprising a module for constructing at least one visual representation of the lumen of the at least one blood vessel.

25. The apparatus of claim 22 wherein parts of the at least one blood vessel and the sediment submerged therein are indicated using color-coding.

26. The apparatus of claim 22 further comprising a width determination module for determining:
the width of the sediments layers at a location along the at least one blood vessel; and
the diameter of the at least one blood vessel at a location along the blood vessel; and
the percentage of stenosis of the at least one blood vessel at a location along the blood vessel.

27. The apparatus of claim 26 where the widths of the sediment layers, the diameter of the blood vessel and the percentage of stenosis are indicated on the at least one second image.

28. The apparatus of claim 22 further comprising a module for indicating, in response to a user action, at least one part of an at least one blood vessel as non-flexible.

29. The apparatus of claim 22 further enabling comprising a module for indicating, in response to a user action, at least one part of an at least one blood vessel as curved.

30. The apparatus of claim 22 further comprising a check-point definition module for indicating, in response to a user action, a position within the body of a patient as a check-point and associate said check-point with an at least one second image, or an at least one set of perspectives for the medical imaging device employed during the operation.

31. The apparatus of claim 22 where the at least one second image depicts a three-dimensional view of the at least one part of the at least one blood vessel.

32. The apparatus of claim 22 where the at least one second image depicts an at least one surface within the human body and an at least one blood vessel on said surface.

33. The apparatus of claim 22 where the at least one second image depicts an at least one internal three-dimensional view of the at least one blood vessel.

34. The apparatus of claim 22 where the at least one second image depicts a cross-section of the at least one blood vessel at a location along the blood vessel, said cross-section comprising one or more of the following: the blood vessel's wall, the lumen of the blood vessel, sediments submerged on the blood vessel's wall.

35. The apparatus of claim 22 further comprising a module for manually correcting the indications for sediments on images acquired prior to an operation and the products of said images.

36. The apparatus of claim 35 where the correction includes changing the size or the sediment type of the indication, adding, or deleting indications.

37. The apparatus of claim 22 where the blood vessel is a coronary artery.

38. The apparatus of claim 22 where the sediments are any one of the following: lipid-rich plaque, intermediate plaque, calcified plaque, thrombi, cells or products of cells.

39. The apparatus of claim 22 where the medical imaging device is a multi slice computerized tomography device.

40. A method for displaying an at least one first image, said first image is a product of processing at least one second image taken by a first medical imaging device prior to a medical operation, the image comprising information about areas with sediments, during the operation.

41. The method of claim 40 where the at least one first image is fused with an at least one third image which is a product of processing an at least one fourth image taken by a second medical imaging device during the operation, the method comprises the following steps:
registering the coordinate systems of the first and the third images; and
fusing information contained in and associated with the at least one first image and the at least one third image; and
presenting an at least one combination image, the combination image is selected from the group consisting of: the at least one first image, the at least one third image, a combination of the at least one first and the at least one third images containing information obtained from the at least one first or the at least one second medical imaging devices, an image containing information obtained from the first and second medical imaging devices.

42. The method of claim 41 where the registration of the coordinate systems comprises the following steps:
global registration of the first image and the third image;
removal of local residual discrepancies by matching corresponding features detected in the first and the third images.

43. The method of claim 42 where the global registration is based on comparing the coordinates of at least one fiducial as seen in the first and the third images.

44. The method of claim 42 where the global registration is based on matching an at least one third image to an at least one projection of a three dimensional data obtained from the at least one first image prior to the operation.

45. The method of claim 41 further comprising a step of correcting an at least one imaging error in the at least first image, using the at least one third image

46. The method of claim 45 where the at least one imaging error is characterized by an at least one calcified area of an at least one blood vessel depicted outsized on an at least one product of processing of at least one image taken by a medical imaging device prior to an operation.

47. The method of claim 41 where the at least one first image and the at least one third image, are presented on the same location on the visual display, where the first and the third images are at least partially transparent.

48. The method of claim 41 where at least one part of the at least one first image, and at least one part of the at least one third image are presented adjacent to each other.

49. The method of claim 41 where sediments found by processing the at least one first image are marked on the at least one third image.

50. The method of claim 41 further comprising a step of marking at least one non-flexible part of an at least one blood vessel, on the at least one first image.

51. The method of claim 41 further comprising a step of marking at least one non-flexible part of an at least one blood vessel, on the at least one third image.

52. The method of claim 41 further comprising a step of marking at least one curved portion of an at least one blood vessel, on the at least one first image.

53. The method of claim 41 further comprising a step of marking at least one curved portion of an at least one blood vessel, on the at least one third image.

54. The method of claim 41 further comprising the steps of:
identifying at least one point in an at least one image acquired during the operation with an at least one check-point indicated prior to the operation; and
presenting at least one image associated prior to the operation with the at least one check-point.

55. The method of claim 40 where the blood vessel is a coronary artery.

56. The method of 40 where the sediments are lipid-rich plaque, intermediate plaque, calcified plaque, thrombi, cells or products of cells.

57. The method of 40 where the medical imaging device is a multi-slice computerized tomography device.

58. A method for automatically detecting an at least one area of an at least one blood vessel having sediment submerged thereto, said sediment is of one or more types, using at least one first image acquired by an at least one medical imaging device, the method comprising the steps of:
identifying the at least one area of the at least one blood vessel with sediments layers; and
indicating the at least one area on an at least one second image, said second image is depicting a product of processing the at least one first image.

59. The method of claim 58 where the identification step comprises the step of comparing the intensity value of an at least one pixel of an at least one image acquired by a medical imaging device to at least one range of intensity values for an at least one type of sediment.

60. The method of claim 58 further comprising the step of constructing an at least one visual representation of the lumen of the at least one blood vessel;

61. The method of claim 58 where sediments submerged in the at least one blood vessel are indicated using color-coding.

62. The method of claim 58 further comprising the steps of determining any one of the following:
the width of the sediment layers at a position along the at least one blood vessel; and
the diameter of the blood vessel at a position along the at least one blood vessel; and
the percentage of stenosis of the at least one blood vessel at a position along the blood vessel.

63. The method of claim 62 further comprising the step of indicating on the second image the at least one width of the sediments layers, the diameter of the at least one blood vessel and the percentage of stenosis.

64. The method of claim 58 further comprising a step of marking on the second image, in response to a user's actions, at least one part of an at least one blood vessel as non-flexible.

65. The method of claim 58 further comprising a step of marking on the second image, in response to a user's actions, at least one part of an at least one blood vessel as being curved.

66. The method of claim 58 further comprising a step of indicating on the second image, in response to a user's actions, a point within the body of a patient as a check-point and associate said check-point with an at least one image, said image is a product of processing images taken by a medical imaging device prior to an operation, or an at least one set of perspectives for the medical imaging device employed during the operation.

67. The method of claim 58 wherein the at least one second image depicts an at least one three-dimensional view of the at least one blood vessel.

68. The method of claim 58 where the at least one second image depicts an at least one three-dimensional surface within the human body.

69. The method of claim 58 where the at least one second image depicts an internal three-dimensional view of a coronary artery.

70. The method of claim 58 where the at least one second image depicts a cross-section of the at least one blood vessel, at a location along the at least one blood vessel, said cross-section comprising any one of the following: the blood vessel wall, the lumen of the blood vessel, sediment.

71. The method of claim 58 further comprising the step of providing a user with the option to manually correct the indications for sediments on images acquired prior to an operation and on the products of processing said images.

72. The method of claim 58 where the correction includes any one of the following: changing the size or the sediment type of an indication, adding, or deleting indications.

73. The method of claim 58 where the blood vessel is a coronary artery.

74. The method of claim 58 where the sediments are lipid-rich plaque, intermediate plaque, calcified plaque, thrombi, cells or products of cells.

75. The method of claim 58 where the medical imaging device is a multi slice computerized tomography device.

76. A method for automatic reconstruction of at least one three-dimensional object from two angiograms using CT information, the method comprises the following steps:
taking a first and a second angiograms of a required area from different perspectives; and
for the first and the second angiogram, obtaining a first and a second projected CT images by projecting the three dimensional CT data on the same plane as the first and the second angiogram; and
registration of the first and the second angiogram with the corresponding projected CT images by objects appearing in the first or second angiogram and in the first or second projected CT, respectively; and
mutual co-registration of the first and the second angiograms; and
detecting at least one object appearing in the angiogram and matching with a corresponding object in the projected CT; and
deriving the three dimensional coordinates of an at least one object appearing in the first and the second angiograms; and
constructing a three dimensional image of the required area from the first and the second angiogram.
